(19) United States
(12) Patent Application Publication
(10) Pub. No.: US 2023/0181357 A1
(43) Pub. Date: Jun. 15, 2023

(54) IMPLANTABLE DRUG DELIVERY DEVICE WITH A SELF-SEALING RESERVOIR FOR TREATING OCULAR DISEASES

(71) Applicant: InnFocus, Inc., Miami, FL (US)

(72) Inventors: Leonard Pinchuk, Miami, FL (US); John B. Martin, Miami, FL (US)

(73) Assignee: InnFocus, Inc., Miami, FL (US)

(21) Appl. No.: 17/923,893

(22) PCT Filed: Feb. 10, 2021
(86) PCT No.: PCT/US2021/017412

§ 371 Date: Nov. 7, 2022

Related U.S. Application Data
(60) Provisional application No. 62/976,543, filed on Feb. 14, 2020.

(51) Int. Cl. A61F 9/00

(52) U.S. Cl. CPC A61F 9/0017; A61F 2250/0068; A61F 2250/0069

(57) ABSTRACT

Implantable devices and systems and methods are provided for controlled delivery of a therapeutic agent to the eye, which employ a flexible reservoir for holding a supply of the therapeutic agent, and a flexible tube that extends from the reservoir. The tube has an inlet end in fluid communication with the interior space of the reservoir, an outlet end spaced from the reservoir, and a lumen that extends from the inlet end to the outlet end. The lumen of the tube is configured to deliver therapeutic agent from the reservoir through the tube.

### BACKGROUND

### 1. Field

The present disclosure relates to implantable drug delivery devices and systems and methods for treating ocular diseases.

### 2. State of the Art

In order to treat certain ocular diseases, there is a need to provide a constant infusion of a liquid-form therapeutic agent (or drug) within the eye. For example, in the treatment of wet macular degeneration, the patient undergoes monthly injections of the liquid form agent Bevacizumab (which is sold under the trade name “Avastin®”), which is an anti-VEGF drug to stop the overgrowth of the macula with blood vessels. This monthly injection is painful to the patient and bothersome to the medical providers who inject the drug. In addition, there is a risk of infection every time a needle is inserted into the eye.

### SUMMARY

The present disclosure describes an implantable drug delivery device for treating ocular diseases that includes a self-sealing reservoir that can be loaded to hold a volume of a liquid-form therapeutic agent. The device further includes a tube that extends from the reservoir. The device can be implanted in the eye where all or parts of the device are surrounded and covered by ocular tissue with the free end of the tube located in a desired position. The opposite end of the tube is in fluid communication with the interior space of the reservoir. The tube can be configured to provide outflow of the liquid-form therapeutic agent held in the reservoir through the tube for discharge out the free end of the tube. Furthermore, a hollow syringe needle connected to a syringe can be used to load (e.g., fill or refill) the reservoir with the liquid-form therapeutic agent in this implanted configuration. In this configuration, the syringe can be configured to hold the therapeutic agent and operated to pump therapeutic agent through the hollow syringe needle into the reservoir. The reservoir and tube can be configured to provide a desired outflow (delivery) of the therapeutic agent through the tube, such as a flow rate that continues over a desired period of time (for example, a period of time in weeks to years). In the implanted configuration, the needle can be used to load the reservoir with the therapeutic agent as needed, such as when the discharge of the therapeutic agent out the free end of the tube stops or falls below a desired level and/or the therapeutic agent is depleted in the reservoir. Drug delivery systems for treating ocular diseases as described herein can include the drug delivery device with the reservoir of the device holding liquid-form therapeutic agent.

In embodiments, the device can be implanted in the eye with the free end of the tube located within the anterior chamber or posterior chamber of the eye. Furthermore, the reservoir can be loaded (e.g., filled, or refilled) with the liquid-form therapeutic agent in this implanted configuration in order to deliver the liquid-form therapeutic agent held in the reservoir through the tube for discharge out the free end of the tube and into the anterior chamber or posterior chamber of the eye.

The device and system can be used to treat wet macular degeneration where the reservoir is loaded with the liquid form agent Bevacizumab and the tube delivers the liquid form agent Bevacizumab held in the reservoir to the posterior chamber of the eye. The device and system can be used to treat other ocular diseases such as glaucoma where the reservoir is loaded with prostaglandins, beta blockers and the like and the tube delivers such liquid form agents held in the reservoir to the anterior chamber or posterior chamber of the eye. The device and system can be used to treat other ocular diseases such as uveitis where the reservoir is loaded with a liquid-form anti-inflammatory agent such as dexamethasone and the like and the tube delivers such liquid form agents held in the reservoir to the anterior chamber or posterior chamber of the eye. The device and system can be used to treat other ocular diseases or disorders where the reservoir is loaded with one or more liquid-form agents that compensate for or treat genetic abnormalities in the eye and the tube delivers such liquid form agents held in the reservoir to the anterior chamber or posterior chamber of the eye. The reservoir and tube can be configured to provide a desired outflow (delivery) of the therapeutic agent through the tube, such as a flow rate that continues over a desired period of time (for example, a period of time in weeks to years).

### BRIEF DESCRIPTION OF THE DRAWINGS

FIG. 1A shows a schematic of an embodiment of a drug delivery device, referred to herein as a device or system or “DDS”, in accordance with the present disclosure.

FIG. 1B shows an alternative embodiment of a DDS in accordance with of the present disclosure.

FIG. 2 shows a top view of the DDS of FIG. 1A with the reservoir being oval shaped.

FIG. 3 shows the DDS of FIG. 1A at an exemplary implantation site in the eye where the reservoir is implanted at a location under the conjunctiva and Tenon's Capsule in the eye and the contoured base of the DDS sits on the sclera of the eye; the tube of the DDS is in fluid communication with the interior space of the reservoir and its free end extends into the anterior chamber of the eye.

FIG. 4 shows the DDS of FIG. 1A at an implantation site similar to FIG. 3; however, the tube of the DDS is in fluid communication with the interior space of the reservoir and its free end extends into the posterior chamber of the eye.

FIGS. 5A, 5B and 5C show a prototype DDS (similar to the DDS of FIG. 1A and the DDS of FIG. 1B) implanted in a rabbit eye.

FIG. 6A shows another embodiment of a DDS in accordance with of the present disclosure.

FIG. 6B is an exploded view of part of the DDS of FIG. 6A.

FIG. 6C is a partial cross-sectional view of part of the DDS of FIG. 6A.

### DETAILED DESCRIPTION

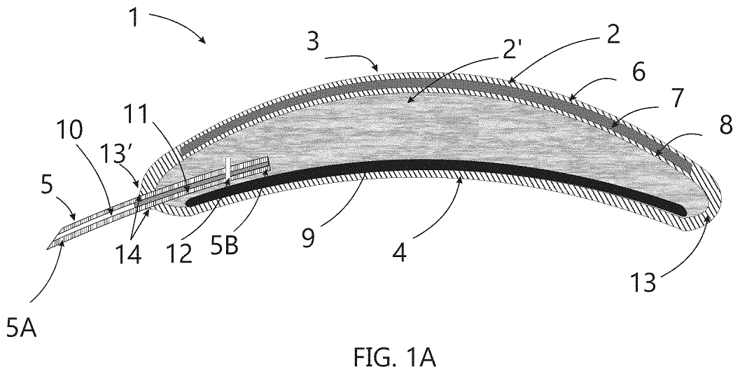

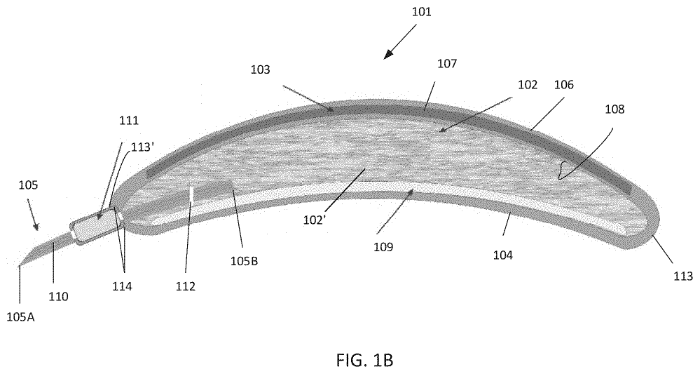

FIG. 1A shows a schematic of a drug delivery device or system (DDS) 1, which includes a fluid reservoir 2 formed by a self-sealing polymeric membrane 3 and a base 4. The base 4 can have a bottom concave surface that is contoured to interface and rest naturally in an implanted configuration on ocular tissue that forms the globe of the human eye. A drug delivery tube 5 extends from the reservoir 2. The DDS 1 can be implanted in the eye where all or parts of the DDS 1 are surrounded and covered by ocular tissue with the outflow end 5A of the tube 5 located in a desired position. The opposite inflow end 5B of the tube is in fluid communication with the interior space 2′ of the reservoir 2. In embodiments, a portion of the tube that includes the inflow end 5B can be coiled within the interior space 2′ of the reservoir 2. The tube 5 has a lumen 10 that extends along the entire length of the tube 5 between its ends 5A and 5B. In use, the interior space 2′ of the reservoir 2 can be configured to hold a supply of a liquid-form therapeutic agent, and the lumen 10 of the tube 5 delivering such therapeutic agent through the tube 5 from the inflow end 5B to the outflow end 5A as described herein.

The self-sealing polymeric membrane 3 can be formed of a three-layer polymeric laminate structure which includes a middle polymer layer 7 sandwiched between an outer polymer layer 6 and an inner polymer layer 8 as shown in FIG. 1A. In embodiments, the middle polymer layer 7 is formed of a polymeric material that is softer (lower durometer) than the outer polymer layer 6 and the inner polymer layer 8. For example, the middle polymer layer 7 can be realized from a SIBS polymer of Shore 10A to 30A (preferably Shore 20A), while the outer polymer layer 6 and the inner polymer layer 8 can be realized from a SIBS polymer of Shore 30A to 60A (preferably Shore 40A). The three-layer laminate polymeric structure can be integrally formed by solvent casting or by heat-fusing the three polymer layers (6, 7, 8) together in a compression mold machine (for example, at 310 to 360° F., 5,000-20,000 psi for 2-5 minutes).

The three-layer laminate polymeric structure of the self-sealing membrane 3 is configured to be pierced by a needle in order to load (e.g., fill and/or refill) the interior space 2′ of the reservoir 2 with the desired liquid-form therapeutic agent. During this process, the harder and stiffer polymer layers 6 and 8 hold the softer middle polymer layer 7 in rigid proximity. When the needle is inserted through the three-layer laminate polymeric structure and then removed, the softer middle polymer layer 7 quickly recoils back to its original position and effectively seals the needle tract thereby preventing fluid held in the fluid reservoir 2 from escaping out through the needle tract. A drug delivery system for treating ocular diseases can include the DDS 1 of FIG. 1A with the reservoir 2 of the DDS 1 holding liquid-form therapeutic agent.

In alternate embodiments, the inner polymer layer 8 can be omitted from the self-sealing membrane 3. It is also possible to repeat the three layer (or two layer) structure as part of the self-sealing injection membrane 3 by laminating the polymer layers together. It is also possible that the outer polymer layer 6 can be made from the softer polymer material with an underlying layer of harder polymer material or that the self-sealing injection membrane 3 can be formed from a single polymer layer. In all of these configurations, when a needle is inserted through the self-sealing membrane 3 and removed, the polymeric material of the membrane 3 effectively seals the needle tract thereby preventing fluid held in the fluid reservoir 2 from escaping out through the needle tract. Lastly, although SIBS is used as the example, the materials can be made from silicone rubber or other suitable polymeric material. SIBS is a polyolefinic copolymer material having a triblock polymer backbone comprising polystyrene-polyisobutylene-polystyrene—or poly(styrene-block-isobutylene-block-styrene). High molecular weight polyisobutylene (PIB) is a soft elastomeric material with a Shore hardness of approximately 10A to 30A. When copolymerized with polystyrene, it can be made at hardnesses ranging up to the hardness of polystyrene, which has a Shore hardness of 100D. Thus, depending on the relative amounts of styrene and isobutylene, the SIBS copolymer can have a range of hardnesses from as soft as Shore 10A to as hard as Shore 100D. In this manner, the SIBS copolymer can be adapted to have the desired elastomeric and hardness qualities. Details of the SIBS copolymer is set forth in U.S. Pat. Nos. 5,741,331; 6,102,939; 6,197,240; 6,545,097, which are hereby incorporated by reference in their entirety. Note that SIBS is preferably used for the DDS 1 as it is biocompatible, soft, atraumatic, bioinert and has proven history in the eye greater than 10-years in duration.

The base 4 can be formed from one or more polymer layers with a thin hard needle stopper feature 9. The needle stopper feature 9 can be placed on or bonded to the inside surface of the base 4 or possibly formed as part of the base 4. The polymer layer(s) of the base 4 can be realized from SIBS, silicon rubber or other suitable polymeric material. The needle stopper feature 9 can be realized from a metal (such as titanium or stainless steel) or a hard plastic (such as polyimide, polyacetal or polysulfone) that does not interfere with medical imaging technologies, such as Mill. In one embodiment, the needle stopper feature 9 can be formed from titanium of 0.001 inches thickness. Titanium is used here due to its well-established history in the body and its lack of interference with Mill. When using the needle to load (e.g., fill or refill) the reservoir, the needle stopper feature 9 prevents the needle that pierces the membrane 3 from entering into and passing through the base 4 and possibly injuring the eye that underlies the base 4 as well as providing a pin-hole where liquid-form therapeutic agent can escape. In an alternate embodiment, the base 4 can be formed of a relatively hard material, for example SIBS copolymer of Shore 60D-70D durometer and allow for elimination of the needle stopper feature 9 from the DDS 1. In this configuration, the harder material of the base 4 can resist puncture by the needle.

The tube 5 can have an outer diameter ranging from 0.2 to 1.0 mm (preferably 0.4 mm). The lumen 10 can have a diameter ranging from 50 to 200 μm (preferably 70 μm). The length of the tube 5 can vary by design and will depend upon where it is placed and the desired rate of flow of the liquid-form therapeutic agent through the lumen 10. Further, the tube and tube lumen need not be of uniform diameter down its length; for example, it may be desirable at times that the section of tube 5 that is penetrating tissue be made smaller than the remainder of tube 5 so as to be less traumatic to the tissue.

In embodiments, at least part of the tube 5 that is disposed within the interior space 2′ of the reservoir 2 space can be configured to encapsulate a plug 11. The plug 11 occupies the lumen 10 of the tube 5 and is configured to allow a controlled rate of flow of the liquid-form therapeutic agent held in the interior space 2′ of the reservoir 2 through the lumen 10 for discharge from the outflow end 5A of the tube 5. In embodiments, the plug 11 is formed from a permeable material, such as a hydrogel polymer. Suitable hydrogel polymers include, but are not limited, to Poly(2-hydroxyethyl methacrylate) (“pHEMA”), polyacrylamide, polymethylacrylamide, polymethacrylic acid, polyvinyl acetate, or other hydrogels or combinations of the above or combinations of the above with more hydrophobic polymers such as polymethylmethacrylate or polystyrene, etc.

In embodiments, the liquid-form therapeutic agent held in the interior space 2′ of the reservoir 2 can flow through the lumen 10 for discharge from the outflow end 5A of the tube 5 into the target location in the eye (e.g., anterior chamber or posterior chamber) by passive diffusion or osmosis where the molecules of the therapeutic agent move through the tube 5 from a volume of higher concentration of such molecules in the interior space 2′ of the reservoir 2 to a volume of lower concentration of such molecules at the target location in the eye. Conversely, molecules of the ocular fluid at the target location in the eye (e.g., aqueous humor in the anterior chamber or posterior chamber) can flow through the lumen 10 from the outflow end 5A to the inflow end 5B of the tube 5 and into in the interior space 2′ of the reservoir 2 by diffusion or osmosis where the molecules of the ocular fluid moves through the tube 5 from a volume of higher concentration of such molecules at the target location in the eye to a volume of lower concentration of such molecules in the interior space 2′ of the reservoir 2. The diffusion or osmosis of the therapeutic agent through the plug 11 and tube 5 is dependent on the nature of the therapeutic agent and the nature of the ocular fluid and the nature of the material of the plug (e.g., the effective diffusion coefficient of the therapeutic agent in the ocular fluid across the plug), the cross-sectional diameter and length of the plug 11, and the cross-sectional diameter and length of the lumen 10 of the tube 5. Once the target location and associated ocular fluid, the therapeutic agent, and the material for the plug 11 are established, the rate of diffusion of the therapeutic agent through the plug 11 and tube 5 can be controlled by the length of the plug 11 in the tube 5, the cross-sectional diameter of the plug 11, the diameter of lumen 10, and the length of the lumen 10. In embodiments, the diameter of lumen 10 can control the cross-sectional diameter of the plug 11. In embodiments, the plug 11 (e.g., hydrogel) can be polymerized inside the lumen 10 of the tube 5 to provide a biostable diffusive media to retard and control the rate of diffusion of the liquid-form therapeutic agent held in the interior space 2′ of the reservoir 2 through the lumen 10 of the tube 5. Alternatively, the plug 11 (e.g., hydrogel) can be polymerized in a mold, removed from the mold, and then swollen in water to remove impurities. Once, cleaned, the plug can be dehydrated to a size that can be inserted into the tube 5 and then reswollen to remain encapsulated in the tube 5. Other suitable permeable materials can be similarly configured as part of the plug 11. Alternatively, the plug 11 can be placed in the lumen 10 of the tube 5 (e.g., as a line fit) and glued in place to ensure that fluid does not circumvent the plug in the Tube 5. Appropriate glues can include cyanoacrylate, epoxies, fibrin glue and the like.

In other embodiments, the liquid-form therapeutic agent can flow from the interior space 2′ of the reservoir 2 through the lumen 10 for discharge from the outflow end 5A of the tube 5 into the target location in the eye (e.g., anterior chamber or posterior chamber) by pressurization of the therapeutic agent in the interior space 2′ of the reservoir 2. In this case, the therapeutic agent in the interior space 2′ of the reservoir 2 can be pressurized to a higher pressure relative to the pressure of the ocular fluid at the target location in the eye such that this pressure differential causes the therapeutic agent to flow from the interior space 2′ of the reservoir 2 through the lumen 10 for discharge from the outflow end 5A of the tube 5 into the target location in the eye. Such pressurization can possibly be applied by operation of a hollow syringe needle and syringe that is used to load or fill the interior space 2′ of the reservoir 2 with therapeutic agent as described herein. Alternatively, such pressurization can be applied by manual application of compressive forces to the reservoir 2 when it is loaded with therapeutic agent. It is contemplated that such pressurization can be used to quickly deliver a dose of the therapeutic agent to the target location in the eye as needed. Furthermore, the quantity or dose of the therapeutic agent delivered to the target location in the eye can be limited by the volumetric capacity of the therapeutic agent loaded into the interior space 2′ of the reservoir 2, and can be regulated or selected by controlling the pressurization of the therapeutic agent in the interior space 2′ of the reservoir 2.

In other embodiments, the plug 11 need not be part of the DDS 1 and thus can be avoided. In this case, diffusion of the therapeutic agent through the tube 5 is dependent on the nature of the therapeutic agent and nature of the ocular fluid (e.g., the diffusion coefficient of the therapeutic agent in the ocular fluid), and the cross-sectional diameter and length of the lumen 10 of the tube 5. Furthermore, the therapeutic agent can flow from the interior space 2′ of the reservoir 2 through the lumen 10 for discharge from the outflow end 5A of the tube 5 into the target location in the eye (e.g., anterior chamber or posterior chamber) by pressurization of the therapeutic agent in the interior space 2′ of the reservoir 2 as described herein.

In embodiments, the liquid-form therapeutic agent can flow from the interior space 2′ of the reservoir 2 through the lumen 10 for discharge from the outflow end 5A of the tube 5 into the target location in the eye (e.g., anterior chamber or posterior chamber) by pressurization followed by diffusion or osmosis as described herein, by diffusion or osmosis followed by pressurization as described herein, or by other operational sequences that involve pressurization and diffusion or osmosis as described herein.

In embodiments, the tube 5 can be configured to dampen pressure spikes applied to the interior space 2′ of the reservoir 2, which can cause spikes in flow of the therapeutic agent through the tube 5 into the target location in the eye (e.g., anterior chamber or posterior chamber). For example, pressure spikes can be applied to the interior space 2′ of the reservoir 2 by compressive forces applied to the reservoir 2 when a patient rubs his or her eyes. More particularly, the elastomeric properties of the tube 5 can permit for diametric expansion or contraction of the annular wall of the tube 5 in response to a pressure spike where the diametric expansion effectively absorbs and dampens the pressure spike. Such diametric expansion or contraction can occur over a lengthwise segment of the tube 5 that is contained inside the interior space 2′ of the reservoir 2 and/or over a lengthwise segment of the tube 5 that is contained outside the interior space 2′ of the reservoir 2.

In an alternate embodiment (not shown), the entrance into the lumen 10 of the tube 5 in the reservoir 2 can be configured as a duck-billed valve, which consists of a short segment of the tube (1-2 mm) being compressed flat while still maintaining a lumen. In this configuration, if the reservoir 2 is pressurized by pressure spike by rubbing one's eye, or the like, the flattened entrance to the tube will compress closed to effectively prevent flow from the reservoir 2 into the tube. Alternatively, a similar valve-like action can be effectuated by making a segment of the tube 5 in the reservoir 2 thin-walled such that a pressure spike collapses the tube 5 and prevents fluid flow within the tube 5.

Furthermore, the polymeric materials of the self-sealing membrane 3, the base 4 and the tube 5 can be selected to be impervious to the therapeutic agent held within the reservoir 2 and thus prevent diffusion of the therapeutic agent through the walls of the reservoir 2 or through the annular wall along the lengthwise extent of the tube 5.

The self-sealing membrane 3 and the base 4 can be bonded together or otherwise assembled to form the reservoir 2 with a first part of the tube 5 (including outflow end 5A) extending from the reservoir 2 and a second part of the tube 5 (including the inflow end 5B) extending within the interior space 2′ of the reservoir 2. The plug 11 can be disposed within either one or both of the first and second parts of the tube 5 of the DDS 1 as shown in FIG. 1A.

FIG. 1B shows an alternative embodiment of a drug delivery device or system (DDS), where like elements of the embodiment of FIG. 1A are incremented by “100” in FIG. 1B. The DDS 101 includes a relatively larger plug 111 (e.g., hydrogel slug) encapsulated in an enlarged section of tube 105 that is disposed either internal or external to the reservoir 102 (shown external in FIG. 1B) or in a discrete cartridge housing that is fluidly coupled as part of the flow path of the tube 105. The enlarged section of the tube 105 or cartridge is configured to house, encapsulate, and retain the plug 111. The plug 111 may be prefabricated, cleaned, and inserted, glued or not glued, into the enlarged section of the tube 105 or cartridge. In embodiments, the plug 111 is formed from a hydrogel polymer that swells inside the tube 105 or cartridge. A drug delivery system for treating ocular diseases can include the DDS 101 of FIG. 1B with the reservoir 102 of the DDS 101 holding liquid-form therapeutic agent.

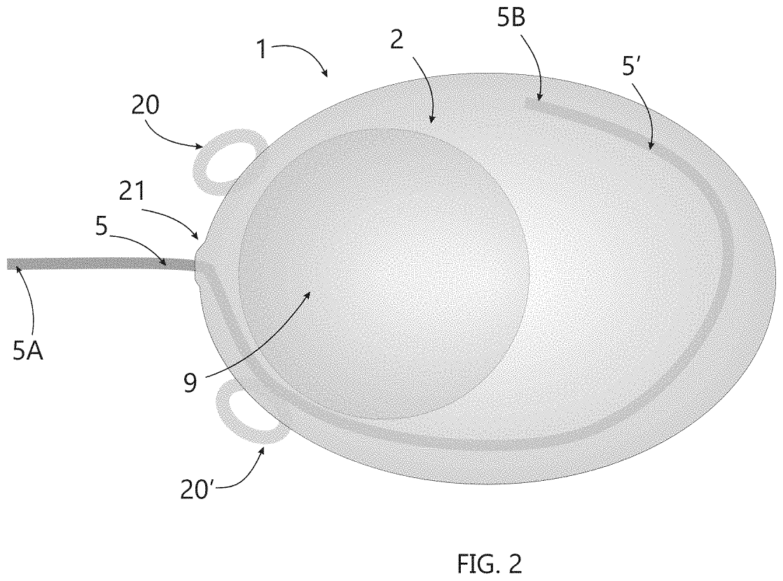

FIG. 2 shows a top view of the DDS 1 of FIG. 1A with the reservoir 2 being oval shaped. In other embodiments, the reservoir 2 can be round or of any shape best suited for implant in the eye. Note that the tube 5 can protrude anywhere from reservoir 2 and need not be along the long axis as is shown in FIG. 2. The outflow end 5A of the tube 5 can be in placed in the anterior chamber or in the vitreous in the posterior chamber of the eye. The lengthwise portion 5′ of the tube 5 can be coiled within reservoir 2 to provide an extended pressure dampening function to the DDS 1 as described above. Note that the needle stopper feature 9 need not cover the entire area of the base 4 and can be located in the vicinity where the needle will be inserted in the eye and into the reservoir 2 as a means of loading the reservoir 2. The DDS 1 can also include fixations structures or ears 20 and 20′ than can aid in fixating the DDS 1 at a desired implantation location in the eye (for example, by suturing through the ears into ocular tissue such as the sclera). In alternate embodiments, the DDS 101 of FIG. 1B can have an oval-shaped reservoir 102 similar to the reservoir 2 of FIG. 1A.

In embodiments, the DDS 1 of FIG. 1A can be made as follows using SIBS as an exemplary material. A thin polymer sheet (which forms the inner polymer layer 8) of SIBS of durometer Shore 40A is cast or compression-molded on a flat surface. A second polymer layer (which forms the middle polymer layer 7) of SIBS of durometer Shore 20A is made separately or cast over the thin polymer sheet. A third polymer layer (which forms the outer polymer layer 6) of SIBS of durometer Shore 40A is made separately or cast over the second polymer layer. Each layer can be 0.001 inches to 0.02 inches in thickness. If made separately, the three layers are stacked on top of one another and compressed in a heated mold (320-360° F., 5,000-20,000 psi for 2-5minutes) thereby fusing the three layers together. The resultant wall thickness is 0.01 to 0.04 inches, preferably approximately 0.02 inches. A first disk is then cut out from this three-layer polymeric structure using a sharp punch. Another polymer membrane (for the base 4) is made using a film of SIBS of durometer Shore 40A. This membrane is approximately the same thickness as the three-layer structure of the first disk. A second disk whose diameters matches the first disk is punched out of this polymer membrane (for the base 4). An assembly comprised of the second disk, a titanium film for the needle stopping feature 9, and the first disk are stacked and placed on a curved metal ball of the same diameter as the human eye, which is approximately 1 inch in diameter. The edges of the assembly are then heat fused together on the ball at or just below the melting point of SIBS which is approximately 180° C. Alternatively, the assembly can be solvent bonded together using a lacquer. The lacquer is preferably made from SIBS 40A dissolved in tetrahydrofuran or toluene (15% solids). The fused edge is represented as 13 and 13′ in FIG. 1A. Heating, during or following assembly of the device, on the ball ensures that the assembly has the correct radius of curvature to rest comfortably on the eye. A hole is then punched in edge 13′ and the tube 5 with plug 11 is inserted and bonded in place with the previously-described lacquer. The lacquer bond is shown as 14 in FIG. 1A. Following bonding the tube 5 to the reservoir 2, the resulting assembly can be dipped into the lacquer, removed, and then dried in an oven at 60° C.-100° C. This procedure rounds all of the edges and assures that the resulting assembly is leakproof. The titanium film (the needle stopping feature 9) can be adhered to the inside surface of the base 4 before assembly, but it need not be adhered as when the therapeutic agent is injected into reservoir 2, the titanium film (the needle stopping feature 9) is forced against inside surface of the base 4. In the embodiment of the DDS 101 shown in FIG. 1B, the fused edge is represented as 113 and 113′. A hole is punched in edge 113′, and the inlet end 105B′ of the tube 105′ is introduced through the hole into the interior space 102′ of the reservoir 102 so that the cartridge or enlarged section of the tube 105 is bonded in place to the edge 113′ with the lacquer. Specifically, one end (closest to inlet end 105B′) of the enlarged section of tube 105 or cartridge is bonded in place to the reservoir 102 at edge 113′. Following bonding the tube 105 to the reservoir 102, the resulting assembly is dipped into the lacquer, removed, and then dried in an oven at 60° C.-100° C. In alternate embodiments, the DDS 101 of FIG. 1B can be constructed in a similar manner to that described above for the DDS 1.

In embodiments, the reservoir 2 of the DDS 1 can be implanted at location under the conjunctiva and Tenon's Capsule in the eye such that the contoured base 4 sits on the sclera of the eye. The radius of curvature of the contour of the base 4 as shown in FIG. 1A is approximately 0.5 inches (12.5 mm). The leading edge 21 can be located close to the limbus of the eye such that the reservoir 2 and the needle stopping feature 9 can easily be seen under an operating microscope for guiding a needle through and into the reservoir 2 for loading the reservoir 2 with the desired therapeutic agent. In alternate embodiments, the DDS 101 of FIG. 1B can be implanted at a similar location in the eye to that described above for the DDS 1.

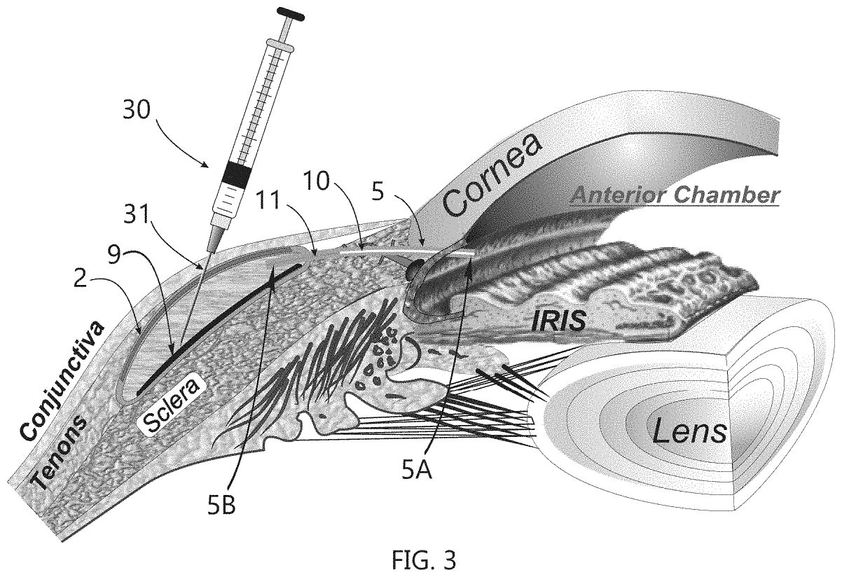

FIG. 3 shows the DDS 1 in an exemplary implantation site in the eye where the reservoir 2 is implanted at location under the conjunctiva and Tenon's Capsule in the eye such that the contoured base 4 sits on the sclera of the eye. The tube 5 is in fluid communication with the interior space 2′ of the reservoir 2 and extends into the anterior chamber of the eye. The optional plug 11 fills the back luminal section of tube 5 in this example; however, the plug 11 can be in any segment of lumen 10. A syringe 30 with a hollow needle 31 is shown loading the interior space 2′ of reservoir 2 of the DDS 1 with a liquid-form therapeutic agent. In this configuration, the syringe 30 can be configured to hold the therapeutic agent and operated to pump the therapeutic agent through the hollow needle 31into the interior space 2′ of the reservoir 2. A tissue passageway through the sclera leading into the anterior chamber of the eye can be formed by an instrument, such as a 27-gauge to 23-gauge syringe needle or the two-step knife described in International Patent Application No. PCT/US17/48431, herein incorporated by reference in it is entirety. Or by a combination of a knife and needle. A part of the tube 5 that extends from the reservoir 2 (including the outlet end 5A) can be inserted into and through this tissue passageway using another instrument, such as forceps or an inserter tool as described in International Patent Application No. PCT/US17/48431, such that the outlet end 5A of the tube 5 is located at a position within the anterior chamber of the eye. In alternate embodiments, the DDS 101 of FIG. 1B can be implanted at a similar location in the eye to that shown in FIG. 3 for the DDS 1.

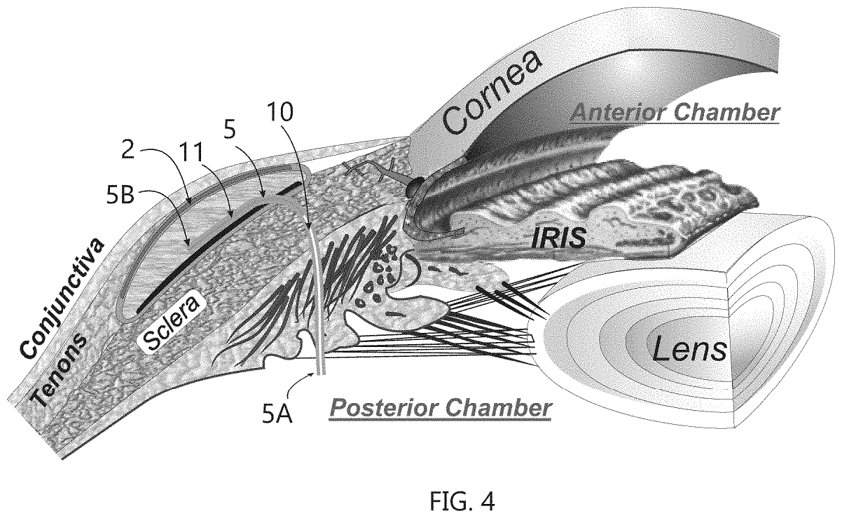

FIG. 4 is similar to FIG. 3, however, the tube 5 is in fluid communication with the interior space 2′ of the reservoir 2 and extends into the posterior chamber of the eye. In this configuration, the tube 5 is oriented such that it is coming out of the edge 13 of the DDS 1 and bypassing the needle stopping feature 9. The optional plug 11 occupies the back section of tube 5 disposed in the interior space 2′ of the reservoir 2 as this length of hydrogel was determined by in vitro testing to be adequate for the desired prolonged release rate of the therapeutic agent from the reservoir 2. A tissue passageway through the sclera leading into the posterior chamber of the eye can be formed by an instrument, such as a 27-gauge to 23-gauge syringe needle or as the two-step knife described in International Patent Application No. PCT/US17/48431, herein incorporated by reference in it is entirety, or by a combination of a knife and a needle. A part of the tube 5 that extends from the reservoir 2 (including the outlet end 5A) can be inserted into and through this tissue passageway using another instrument, such as forceps or an inserter tool as described in International Patent Application No. PCT/US17/48431, such that the outlet end 5A of the tube 5 is located at a position within the posterior chamber of the eye. In alternate embodiments, the DDS 101 of FIG. 1B can be implanted at similar location in the eye to that shown in FIG. 4 for the DDS 1.

In the embodiments of FIGS. 3 and 4, the entire DDS 1 is flexible (including the reservoir 2 with the needle stopper feature 9 as well as the tube 5) such that the reservoir 2 with the needle stopper feature 9 can be folded and/or rolled upon itself. This feature minimizes the size of the incision required for implantation. Particularly, the flexible reservoir 2 with needle stopper 9 can be folded around or with the flexible tube 5 into a compact folded configuration that can fit through a small incision in the conjunctiva. The folded configuration can then be unfolded such that the reservoir 2 with the needle stopper feature 9 rests on the sclera. Furthermore, the flexible tube 5 can bend or buckle under the axial compressive forces that may be imparted by manual forces applied to the tube 5 when the tube 5 is implanted into its desired position in the eye.

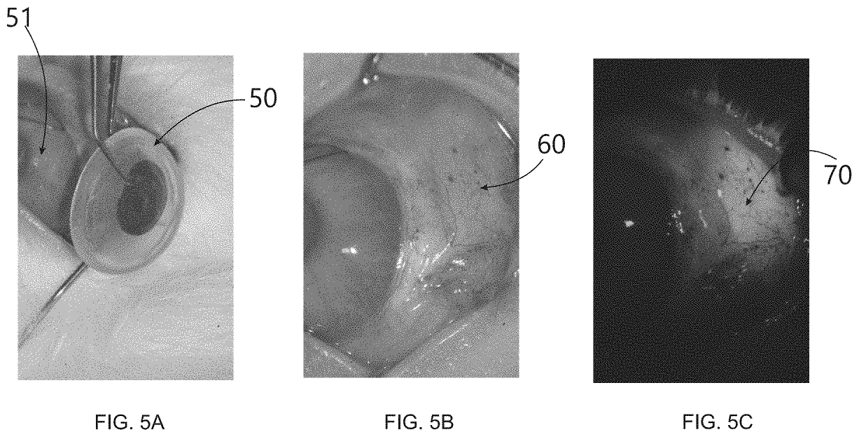

FIGS. 5A, 5B and 5C show a prototype DDS 50 (similar to the DDS 1 of FIG. 1A and the DDS 101 of FIG. 1B as described above) implanted in a rabbit eye 51. The DDS 50 was implanted under the conjunctiva and Tenon's Capsule as illustrated in FIGS. 3 and 4. Then, as photographed in FIG. 5B, the reservoir 2 of the DDS 50 was filled with fluorescein through a 30-G hollow needle that was inserted through the conjunctiva and Tenon's and through the self-sealing membrane 3 of the DDS 50. FIG. 5C shows the fluorescein in the reservoir 2 of the DDS 50 under black light radiation. Applied pressure to the conjunctiva that covers the DDS 50 did not release any fluorescein, thereby confirming the effectiveness of the self-sealing membrane 3 of the DDS 50.

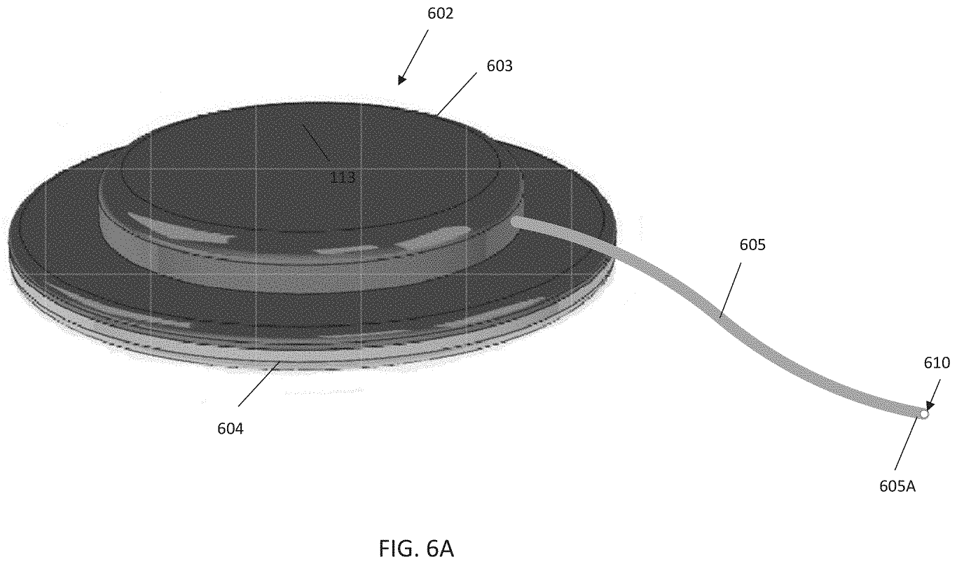

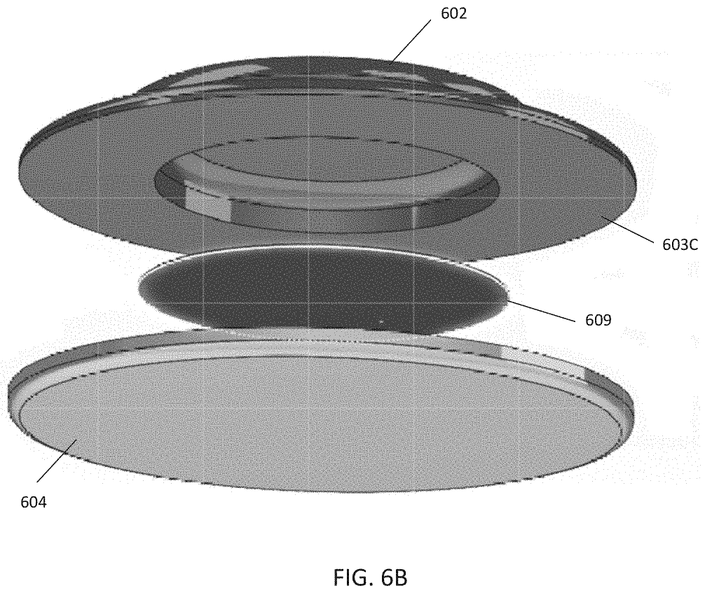

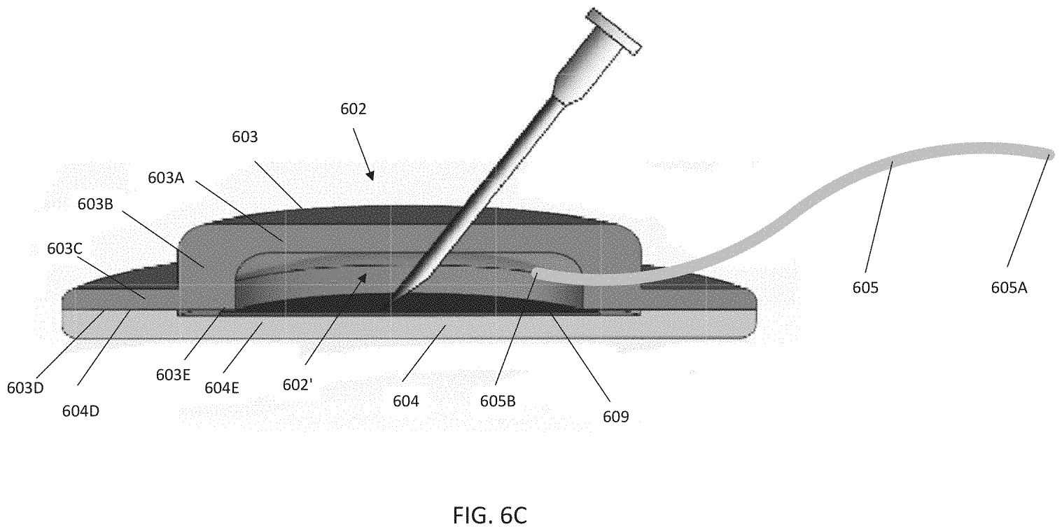

FIGS. 6A, 6B and 6C show another embodiment of a drug delivery device or system (DDS), where like elements in the embodiment of FIG. 1A are incremented by “600” in FIGS. 6A, 6B, and 6C. The DDS 601 of FIGS. 6A, 6B and 6C includes a flexible fluid reservoir 602 formed by a polymeric membrane 603 and a base 604. The base 604 can have a bottom concave surface that is contoured to interface and rest naturally in an implanted configuration on ocular tissue that forms the globe of the human eye. A flexible drug delivery tube 605 extends from the reservoir 602. The DDS 605 can be implanted in the eye where all or parts of the DDS 601 are surrounded and covered by ocular tissue with the outflow end 605A of the tube 605 located in a desired position. The opposite inflow end 605B of the tube 605 is in fluid communication with the interior space 602′ of the reservoir 602 as best shown in FIG. 6C. The tube 605 has an internal lumen 610 that extends along the entire length of the tube 605 between its ends 605A and 605B. In use, the interior space 602′ of the reservoir 602 can be configured to hold a liquid-form therapeutic agent with the lumen 610 of the tube 605 delivering such therapeutic agent through the tube 605 from the inflow end 605B to the outflow end 605A.

In embodiments, the membrane 603 can configured as a top hat structure with a top wall 603A, annular side wall 603B extending downward from the top wall 603A to a bottom flange wall 603C extending outward from the annular side wall 603B as shown in FIG. 6C. The peripheral part 603D of the bottom side of the bottom flange wall 603C is bonded to or otherwise sealed and secured to the opposed peripheral part 604D of the top surface of the base 604. The central part 604E of the base 604 includes a recess that receives a thin hard needle stopper feature 609. The needle stopper feature 609 can be captured or otherwise secured between the central part 603E of the bottom side of the bottom flange wall 603C and the central part 604E of the base 604. The polymer layer(s) of the base 604 can be realized from SIBS, silicon rubber or other suitable polymeric material. The needle stopper feature 609 can be realized from a metal (such as titanium or stainless steel or other metal that does not interfere with medical imaging such as MRI) or a hard plastic (such as polyimide, polyacetal or polysulfone or other hard plastic material that does not interfere with medical imaging such as MM).

The top wall 603A (and possibly other parts) of the membrane 603 can be formed of a self-sealing polymeric laminate structure similar to the self-sealing membrane 3 where the polymeric laminate structure is configured to be pierced by a hollow syringe needle or syringe pump in order to load (e.g., fill and/or refill) the interior space 602′ of the reservoir 602 with the desired liquid-form therapeutic agent as shown in FIG. 6C. When using the hollow syringe needle or syringe pump to load (e.g., fill or refill) the reservoir 602, the needle stopper feature 609 prevents the needle that pierces the top wall 603A from entering into and passing through the base 604 and possibly injuring the eye that underlies the base 604 as well as providing a pin-hole where liquid-form therapeutic agent can escape.

In embodiments, the peripheral part of the bottom flange wall 603C and the peripheral part of the opposed base 604 can include thru-holes or other fixation structures than can aid in fixating the DDS at a desired implantation location in the eye (for example, by suturing through the thru-holes into ocular tissue such as the sclera).

In embodiments, the tube 605 can have an outer diameter ranging from 0.2 to 1.0 mm (preferably 0.4 mm). The lumen 610 can have a diameter ranging from 50 to 200 μm (preferably 70 μm). The length of the tube 605 can vary by design and will depend upon where it is placed and the desired rate of flow of the liquid-form therapeutic agent through the lumen 610. In one embodiment, a length of tube 605 of 10 mm extends from the reservoir 602. Further, the tube and tube lumen need not be of uniform diameter down its length; for example, it may be desirable at times that the section of tube 5 that is penetrating tissue be made smaller than the remainder of tube 5 so as to be less traumatic to the tissue. The cylindrical top hat structure of the membrane 603 can be configured to provide the interior space 602′ of the reservoir 602 with a predefined volume that can vary by design and will depend upon the desired quantity of the liquid-form therapeutic agent to be held in the reservoir 602. In one embodiment, the cylindrical top hat structure of the membrane 603 can be configured to provide the interior space 602′ of the reservoir 602 with a volume of 10 to 100 μliters.

In embodiments, the entire DDS of FIGS. 6A, 6B and 6C (including the reservoir 602 with the needle stopper feature 609 as well as the tube 605) is flexible such that the reservoir 602 with the needle stopper feature 609 can be folded and/or rolled upon itself. This feature minimizes the size of the incision required for implantation. Particularly, the flexible reservoir 602 with needle stopper 609 can be folded around or with the flexible tube 605 into a compact folded configuration that can fit through a small incision in the conjunctiva. The folded configuration can then be unfolded such that the reservoir 602 with the needle stopper feature 609 rests on the ocular tissue at the implantation site. Furthermore, the flexible tube 605 can bend or buckle under the axial compressive forces that may be imparted by manual forces applied to the tube 605 when the tube 605 is placed into the desired location in the eye. A drug delivery system for treating ocular diseases can include the DDS 601 with the reservoir 602 of the DDS 601 holding liquid-form therapeutic agent.

In embodiments, therapeutic agent held in the interior space 602′ of the reservoir 602 can flow through the lumen 610 of tube 605 to the outlet end 605A by diffusion or osmosis and/or pressurization of the reservoir as described herein, or by other means.

In embodiments, the tube 605 need not have an encapsulated plug as described herein. In this case, the flow of therapeutic agent through the lumen 610 of tube 605 by diffusion can be governed by the geometry and length of the tube 605. In other embodiments, the tube 605 can include an encapsulated plug as described herein to provide for control over the flow of therapeutic agent through the lumen 610 of tube 605 by diffusion or osmosis.

In embodiments, the DDS of FIGS. 6A, 6B and 6C can be made as follows using SIBS as an exemplary material. An exemplary DDS is made in the following manner:

1) Four films of SIBS of durometer Shore 50A that are 0.01 inches thick are made by compression molding SIBS powder or pellets in a PTFE-lined compression mold at 160° C. (pressure 15,000 PSI held for 2 minutes).

2) A film of SIBS of durometer Shore 20A that is 0.02 inches thick is made by compression molding SIBS powder or pellets in a PTFE-lined compression mold at 150° C. (e.g., pressure 5,000 PSI held for 2 minutes). The molds are then cooled to room temperature under hydraulic compression and the films released.

3) The SIBS films are then stacked with the Shore 20A SIBS film sandwiched between opposed Shore 50A SIBS films. The SIBS film stack, which is about 0.04 inches thick, is then placed in a compression mold where the SIBS film stack is heated to 160° C. and compressed to a thickness of 0.03 inches.

4) Discs of 0.375 inches in diameter are then punched from the above 0.03 inches thick film and inserted in another compression mold which forms the top-hat 602 of FIG. 6B.

5) The base 604 of FIG. 6B is formed by stacking the other 2 SIBS films of Shore 50A that are 0.01 inches thick and placing this 0.02 inches thick stack in a compression mold where the base takes on the curved form of base 604.

6) The needle stopper 609 is formed from a punched disc of 0.001 inches thick titanium or 0.002 inches thick 316 stainless where the punched disc is 0.3 inches in diameter. The punched disk can then be “domed” using a jeweler's ball and socket doming rig.

7) The base 604, needle stopper 609 and top-hat 602 are then stacked as shown in FIG. 6B and placed on a fusion rig where the top-hat 602 and base 604 flange 603 are fused together at 150° C. using a hot die. The needle stopper 609 remains captured within the reservoir 602.

8) SIBS of Shore 50A hardness is extruded over a 70 μm wire such that the outer diameter of the tube is 0.35 mm.

9) The SIBS tube, still on the wire is inserted in the lumen of a 22-gauge needle and needle is inserted through the wall of the assembled DDS. The SIBS tube is held in place and the 22-gauge needle is withdrawn leaving the SIBS tube penetrating the wall of the DDS.

10) A drop of lacquer comprised of 15% SIBS of Shore 50A durometer dissolved in toluene is placed at the penetration site to seal the penetration site. The wire in the tube is then removed.

11) Holes are then punched along the flange to provide suture anchoring sites when implanted.

The DDS of FIGS. 6A, 6B and 6C can be implanted into the eye as follows. The eye is prepared for conjunctival surgery. A 4 mm long peritomy is made along the limbus and a tract under the conjunctiva and above the sclera is dissected with blunt scissors. Any bleeding vessels in the area are cauterized to maintain hemostasis. The DDS is sutured in place with 9-0 Nylon sutures with its anterior edge placed approximately 6 mm from the limbus. A needle tract is made beginning 3 mm posterior to the limbus and extending into the anterior chamber such that the exit of the needle bisects the angle between the cornea and iris. The SIBS tube on the DDS is inserted into the needle tract with forceps. The conjunctiva is pulled over the DDS and sutured closed.

A syringe is fitted with a 30-gauge hollow needle and the syringe is filled with 100 μL of liquid form therapeutic agent (e.g., prostaglandin). The 30-gauge hollow needle is inserted through the conjunctiva and pierces the top-hat reservoir of the DDS into the interior space of the reservoir where it possibly bottoms out on the needle stopper. The syringe is operated to inject the therapeutic agent into the reservoir of the DDS which causes air to be displaced from the reservoir through the tube causing bubbles to form in the anterior chamber. The injection is discontinued when the bubbles are observed to cease thereby indicating that the reservoir is full. The approximate volume dispensed is 70 μL. The DDS can deliver the therapeutic agent to the anterior chamber (or posterior chamber) of the eye by passive diffusion of the therapeutic agent from the reservoir through the SIBS tube until the intraocular pressure in the eye elevates indicating exhaustion of the reservoir. At this point, the reservoir of the DDS is loaded with a dilute mixture of aqueous humor and therapeutic agent. Another syringe is fitted with a 30-gauge hollow needle, and this 30-gauge hollow needle is then inserted through the conjunctiva and pierces the top-hat reservoir of the DDS into the interior space of the reservoir where it possibly bottoms out on the needle stopper. The syringe is operated to apply suction to aspirate any remaining fluid in the reservoir of the DDS. The syringe is then loaded with 70 μL of therapeutic agent, and the syringe is operated to inject the therapeutic agent through the 30-gauge hollow needle into the reservoir of the DDS, which causes any remnant fluid in the reservoir to flow into the anterior chamber (or posterior chamber) of the eye. In this manner, the DDS is loaded or refilled with the therapeutic agent and rendered effective again.

In other embodiments, the therapeutic agent can be delivered to the anterior chamber (or posterior chamber of the eye) by pressurization of the therapeutic agent in the reservoir of DDS.

The drug delivery devices and systems as described herein can be used to treat ocular disorders where the interior space of the reservoir is loaded with a liquid form therapeutic agent and the lumen of the tube delivers the liquid form agent held in the interior space of the reservoir to a desired location or region or space in the ocular environment. For example, the drug delivery devices and systems as described herein can be used to treat wet macular degeneration where the interior space of the reservoir is loaded with the liquid form agent Bevacizumab and the lumen of the tube delivers the liquid form agent Bevacizumab held in the interior space of the reservoir to the posterior chamber of the eye. The drug delivery devices and systems can be used to treat other ocular diseases such as glaucoma where the interior space of the reservoir is loaded with prostaglandins, beta blockers and the like and the lumen of the tube delivers such liquid form agents held in the interior space of the reservoir to the anterior chamber or posterior chamber of the eye. The drug delivery devices and systems can be used to treat other ocular diseases such as uveitis where the interior space of the reservoir is loaded with a liquid-form anti-inflammatory agent such as dexamethasone and the like and the lumen of the tube delivers such liquid form agents held in the interior space of the reservoir to the anterior chamber or posterior chamber of the eye. The drug delivery devices and systems can be used to treat other ocular diseases or disorders where the interior space of the reservoir is loaded with one or more liquid-form agents that compensate for or treat genetic abnormalities in the eye and the lumen of the tube delivers such liquid form agents held in the interior space of the reservoir to the anterior chamber or posterior chamber or other part of the eye. The reservoir and tube can be configured to provide a desired outflow (delivery) of the therapeutic agent through the tube, such as a flow rate that continues over a desired period of time (for example, a period of time in weeks to years).

There have been described and illustrated herein several embodiments of drug delivery devices and systems and methods of use. While particular embodiments of the invention have been described, it is not intended that the invention be limited thereto, as it is intended that the invention be as broad in scope as the art will allow and that the specification be read likewise. Thus, while particular materials have been disclosed, it will be appreciated that other suitable materials may be used as well. Moreover, while particular configurations have been disclosed in reference to a hydrogel plug it will be appreciated that other configurations could be used as well, that may not require any plug. It will therefore be appreciated by those skilled in the art that yet other modifications could be made to the provided invention without deviating from its spirit and scope as claimed.

1. An implantable device for controlled delivery of therapeutic agent to the eye, comprising:
a flexible reservoir configured to hold a supply of therapeutic agent; and
a flexible tube that extends from the reservoir, the tube having an inlet end in fluid communication with the interior space of the reservoir, an outlet end spaced from the reservoir, and a lumen that extends from the inlet end to the outlet end, wherein the lumen is configured to deliver therapeutic agent from the reservoir through the tube.

2. An implantable device according to claim 1, wherein:
the reservoir has a base contoured to rest on the globe of the eye.

3. An implantable device according to claim 2, wherein:
the reservoir has a self-sealing membrane opposite the base, wherein the self-sealing membrane is configured to automatically seal a needle tract through the membrane formed by a needle for loading the reservoir with the therapeutic agent.

4. An implantable device according to claim 3, wherein:
the self-sealing membrane is formed from a multi-layer polymeric structure.

5. An implantable device according to claim 4, wherein:
the multi-layer polymeric structure comprises SIBS polymers of different durometers.

6. An implantable device according to claim 4, wherein:
the multi-layer polymeric structure comprise an inner layer formed of a first SIBS polymer of a first durometer, a middle layer formed of a second SIBS polymer of a second durometer, and an outer layer formed of a third SIBS polymer of a third durometer, wherein the second durometer is less than the first and third durometers.

7. An implantable device according to claim 2, wherein:
the self-sealing membrane is formed entirely from at least one SIBS polymer.

8. An implantable device according to claim 2, wherein:
the reservoir further includes a needle stopping feature disposed between the self-sealing membrane and the base.

9. An implantable device according to claim 8, wherein:
the needle stopping feature comprises a metal or hard plastic material.

10. An implantable device according to claim 8, wherein:
the needle stopping feature is disposed adjacent to or secured to or integral to the base.

11. An implantable device according to claim 1, wherein:
the flexible reservoir can be folded and/or rolled upon itself.

12. An implantable device according to claim 1, wherein:
both the reservoir and tube comprise at least one SIBS polymer.

13. An implantable device according to claim 1, wherein:
the reservoir and tube are configured such that therapeutic agent held in the interior space of the reservoir flows through the lumen of the tube by pressurization of the reservoir.

14. An implantable device according to claim 1, wherein:
the reservoir and tube are configured such that therapeutic agent held in the interior space of the reservoir flows through the lumen of the tube by diffusion or osmosis.

15. An implantable device according to claim 14, further comprising:
a plug configured to control rate of diffusion of therapeutic agent held in the interior space of the reservoir through the lumen of the tube.

16. An implantable device according to claim 15, wherein:
the plug is encapsulated by a part of the tube.

17. An implantable device according to claim 16, wherein:
the part of the tube that encapsulates the plug is disposed within the interior space of the reservoir.

18. An implantable device according to claim 16, wherein:
the part of the tube that encapsulates the plug is disposed outside the reservoir.

19. An implantable device according to claim 15, wherein:
the plug is part of a cartridge disposed between the inlet end and outlet end of the tube.

20. An implantable device according to claim 15, wherein:
the plug comprises permeable material.

21. An implantable device according to claim 20, wherein:
the permeable material of the plug comprises a hydrogel polymer).

22. An implantable device according to claim 1, wherein:
a lengthwise section of the tube is configured to dampen pressure spikes within the interior space of the reservoir.

23. An implantable device according to claim 1, wherein:
the tube has an outer diameter ranging from 0.2 to 1.0 mm.

24. An implantable device according to claim 1, wherein:
the lumen of the tube has a diameter ranging from 60 to 200 μm.

25. An implantable device according to claim 1, wherein:
a length of the tube of 10 mm extends from the reservoir.

26. An implantable device according to claim 1, wherein:
the reservoir is configured with the interior space having a predefined volume of 10 to 300 μliters.

27. A method for controlled delivery of a therapeutic agent to the eye, comprising:
implanting the device of claim 1 into the eye with the outlet end of the tube positioned at a location in the eye; and
with the device of claim 1 implanted in the eye, loading the flexible reservoir with the therapeutic agent in order to deliver the therapeutic agent through the tube for discharge at the location in the eye.

28. A method according to claim 27, wherein:
the location in the eye lies within the anterior chamber of the eye.

29. A method according to claim 27, wherein:
the location in the eye lies within the posterior chamber of the eye.

30. A method according to claim 27, wherein:
the reservoir is implanted into the eye such that it rests on the sclera of the eye.

31. An implantable device for controlled delivery of a therapeutic agent to the eye, comprising:
a flexible reservoir with a self-sealing membrane that is configured to be pierced by a needle to load the reservoir with the therapeutic agent; and
a flexible tube that extends from the reservoir, the tube having an inlet end in fluid communication with the interior space of the reservoir, an outlet end spaced from the reservoir, and a lumen that extends from the inlet end to the outlet end, wherein the lumen is configured to deliver therapeutic agent from the reservoir through the tube.

32. An implantable device according to claim 31, wherein:
the self-sealing membrane is configured to automatically seal a needle tract through the membrane formed by the needle.

33. An implantable device according to claim 32, wherein:
the reservoir has a base configured to rest on the globe of the eye, and a needle stopping feature disposed between the self-sealing membrane and the base.

34. A system for delivering therapeutic agent to the eye, comprising:
a device according to claim 31, wherein the reservoir of the device holds therapeutic agent for delivery through the tube of the device.

35. A system according to claim 34, further comprising:
a syringe and hollow needle, wherein the syringe is configurable to hold the therapeutic agent; and wherein the hollow needle is configurable to pierce the self-sealing membrane of the device to load the reservoir with therapeutic agent supplied by the syringe.
